(19) United States
(12) Patent Application Publication
(10) Pub. No.: US 2006/0210572 A1
(43) Pub. Date: Sep. 21, 2006

(54) Treatment of lymphatic metastatic cancerous lesions

(76) Inventors: Nicholas Ronald Glover, Oakville (CA); Dimitri Peter Fitsialos, Toronto (CA)

Correspondence Address: BERESKIN AND PARR, 40 KING STREET WEST, BOX 401, TORONTO, ON, M5H 3Y2, CA

(21) Appl. No.: 11/377,186

(22) Filed: Mar. 17, 2006

Related U.S. Application Data
(60) Provisional application No. 60/662,827, filed on Mar. 18, 2005.

(51) Int. Cl. A61K 39/395

(52) U.S. Cl. 424/155.1; 424/178.1

(57) ABSTRACT

Methods for improving the efficacy of cancer therapy are described. In one embodiment the method involves administering the cancer therapeutic to a first cancer lesion wherein the first cancer lesion is connected through the lymphatic system to a second cancer lesion and wherein the cancer therapeutic can thereby travel to the second cancer lesion through the lymphatic system.

This application claims the benefit under 35 USC § 119(e) of U.S. Provisional application No. 60/662,827 filed Mar. 18, 2005 which is incorporated herein by reference.

### FIELD OF THE INVENTION

The invention relates to the treatment of tumor lymphatic metastases using immunotoxins. Specifically, the metastases can be treated by administering the immunotoxin intra- or peritumorally in a primary lesion or in a lesion that is linked to other lesions by lymphatic vessels and that is located ‘up-stream’ of those other lesions.

### BACKGROUND OF THE INVENTION

One of the great challenges in the treatment of cancer is not so much to treat the primary tumor (i.e. the site where the cancer originated) but, rather, to reach and destroy the numerous secondary tumors or metastases. A metastatic lesion can be situated close to the primary tumor or at distant locations, it can be readily accessible, may require invasive surgery to remove it, or, in some cases be located too deeply in the body or be closely associated with anatomical structures that prevent safe surgical removal. In addition, the presence of micrometastases can never be ruled out which leads to the use of preventative removal of apparently tumor free tissue and to the use of regional (radiation) and systemic cancer treatments. Systemic treatments include chemotherapy and, more recently, anti-cancer antibodies and immunotoxins. Other recent approaches include systemically administered agents that activate the immune system so it recognizes and destroy the tumor cells. In a few instances, these immune activators have been administered directly into tumors.

Cancer spreads by invasion of the surrounding tissues, by entering and travelling through the vascular system and/or by invading lymphatic vessels and seeding lymph nodes. It is generally accepted that for most types of cancer, the lymph nodes are usually the first sites for tumor metastases to appear. This is because the architecture of the lymphatic vessels allows for the relatively easy entry of tumor cells—a feature normally utilized by immune cells to enter the lymphatic vessels and travel to the lymph nodes where they participate in immune surveillance and immune responses. As a result, surgical treatment of cancer usually includes the removal of many if not all of the lymph nodes in the lymphatic drainage basin of the primary and secondary tumors. Lymphatic drainage mapping is frequently used to identify the lymphatic structures to be removed along with the tumor, and to subsequently determine the extent of invasion of the cancer. Unfortunately, lymph nodes can be difficult to remove because they are deeply seated or because they are in close proximity to major blood vessels, nerves or other critical anatomical features. This is particularly true of affected lymph nodes that become enlarged and can take on the tissue-invasive property of the primary tumor.

There are many reasons why radiation and/or systemic cancer treatments, such as chemotherapy, fail to control or cure cancer. With respect to systemically administered agents, one factor is the absolute need to balance the administration of an effective dose of the agent with the associated toxicity to normal tissues since the latter are also typically affected by the agent. Because it is given systemically and, thus, becomes quite diluted throughout the body, very large doses are needed to ensure sufficient concentration at the tumor site. Furthermore, the tumor architecture can often prevent the entry of the agent in sufficient amounts to kill the cells. In the case of lymphatic metastases, the systemically administered agent must first leave the circulation, where it becomes further diluted in the interstitial fluid, and must then enter the lymphatic system and reach the affected node(s).

Intratumoral administration of therapeutic agents has been limited to a) agents that activate the immune system such as IL-12 (van Herpen et al., Clin. Cancer Res. 9:2950-2956, 2003), IL-12 plus TNF alpha in microsphere (Sabel et al., Annals of Surg. Oncol. 11:147-156, 2004), adenovirus (Kenzer et al., ASCO 2004 Annual meeting, Abstract 2509), HLA-B7/2 microglobulin gene (Heo DS et al., Human Gene Ther. 9:2031-38), the streptococcal preparation OK-432 (Gochi et al., British J. Cancer 84; 443-451, 2001) or dendritic cells (Song et al., ASCO 2002 Annual meeting, Abstract 1903), b) chemotherapeutic agents in their free form (Osaki et al., Oral Dis. 3:247-253, 1997), in combination with epinephrine in gel formulation (Castro et al., Head & Neck 25:717-31, 2003) or in liposomes (Harrington et al., Clin. Cancer Res. 6: 4939-4949, 2000), or to c) agents to destabilize collagen fibrils surrounding a lesion (Chvapil Anticancer Drug 16: 201-210, 2005). Perilymphatic injection of IL-2 (Cortesina et al., Head & Neck 13:125-131, 1991) were also attempted. There have been no reports of a response in lesions other than those injected with chemotherapeutic, or collagen destabilizing agents. Even with immune activating agents that would have been expected to provide a systemic response to tumors there was only one report of a response that extended beyond the injected lesion where a few locoregional lymph nodes were reduced in size (HLA-B7/2 microglobulin gene; (Heo DS et al., Human Gene Ther. 9:2031-38)).

There is, therefore, a need for a new approach to the treatment of lymphatic metastases that will improve the effectiveness of the treatment of these nodes while minimizing systemic toxicity.

### SUMMARY OF THE INVENTION

The inventors have developed a method to improve the efficacy of cancer therapy. The improved method uses the lymphatic drainage of a cancerous lesion to enable the therapy to reach other lesions. These other lesions can be adjacent but may also lie at a distance from the treated lesion. More specifically, the inventors have shown that by injecting an anti-cancer agent intra-tumorally in a cancerous lesion more than one lesion could be treated at a time.

Accordingly, the present invention provides a method of improving the efficacy of a cancer therapeutic comprising administering the cancer therapeutic to a first cancer lesion wherein the first cancer lesion is connected through the lymphatic system to a second cancer lesion and wherein the cancer therapeutic can thereby travel to the second cancer lesion through the lymphatic system.

In one embodiment, the first cancer lesion can be selected by lymphatic drainage basin mapping to select a tumor that is linked to the largest number of secondary lesions.

Accordingly, in one embodiment, the present invention provides a method of improving the efficacy of the cancer therapeutic comprising: a) conducting lymphatic drainage mapping on a patient; b) selecting a first cancer lesion that is linked through the lymphatic system to one or more second cancer lesions; and c) administering the cancer therapeutic to the first cancer lesion wherein the cancer therapeutic can travel to the second cancer lesions through the lymphatic system.

The present invention also includes kits for carrying out the methods of the inventions. In one embodiment, the kit comprises a cancer therapeutic (such as an immunotoxin) together with instructions for administering to the first cancer lesion. In another embodiment, the kit comprises a cancer therapeutic (such as an immunotoxin) together with instructions for identifying the first cancer lesion. In such an embodiment, the instructions can include instructions for lymphatic mapping as described above.

Other features and advantages of the present invention will become apparent from the following detailed description. It should be understood, however, that the detailed description and the specific examples while indicating preferred embodiments of the invention are given by way of illustration only, since various changes and modifications within the spirit and scope of the invention will become apparent to those skilled in the art from this detailed description.

### BRIEF DESCRIPTION OF THE DRAWINGS

FIG. 1a shows a CT scan for subject #7 of target tumor at screening, at week 4 (end of 4 week dosing period) and week 8 (4 weeks after last dose) showing complete resolution of tumor by week 8.

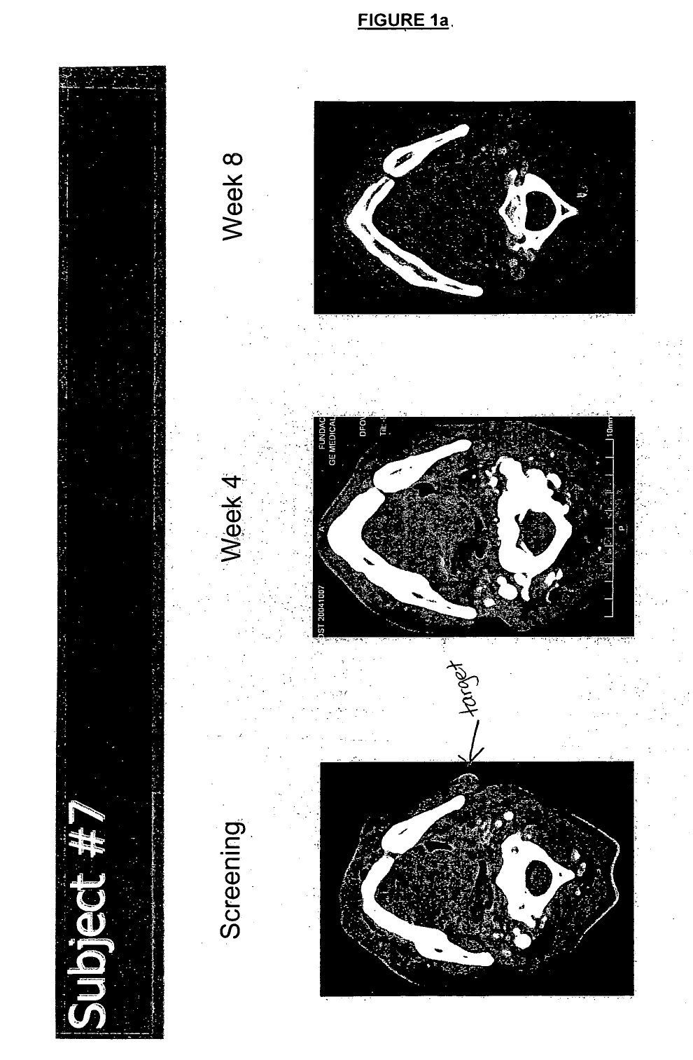

FIG. 1b shows a CT scan for patient #7 of a large deep secondary tumor at screening, at week 4 (end of 4 week dosing period), week 8 (4 weeks after last dose) and 1 month after last planned visit (approximately week 12) showing a decrease in the size of the tumor.

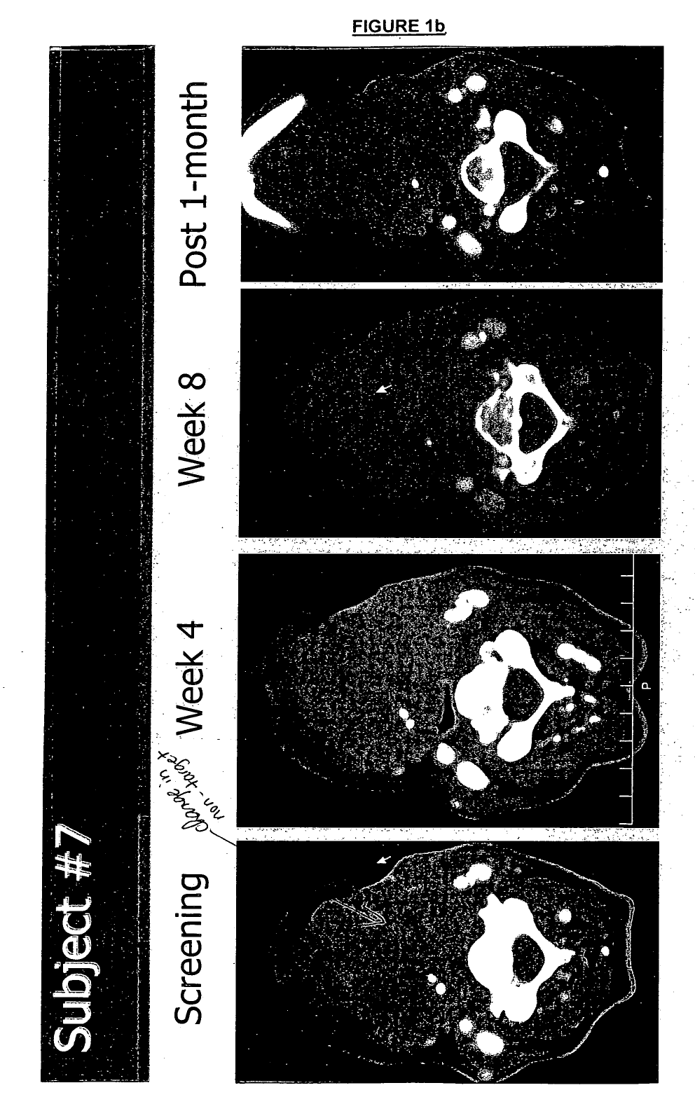

FIG. 2 shows a CT scan for subject #17 of target tumor (large scan) and a secondary tumor (lower small scan) at a screening, at week 4 (end of 4 week dosing period) and week 8 (4 weeks after last dose) showing partial response in target tumor and complete resolution of a secondary tumor. Partial response in other lesions reported but not shown.

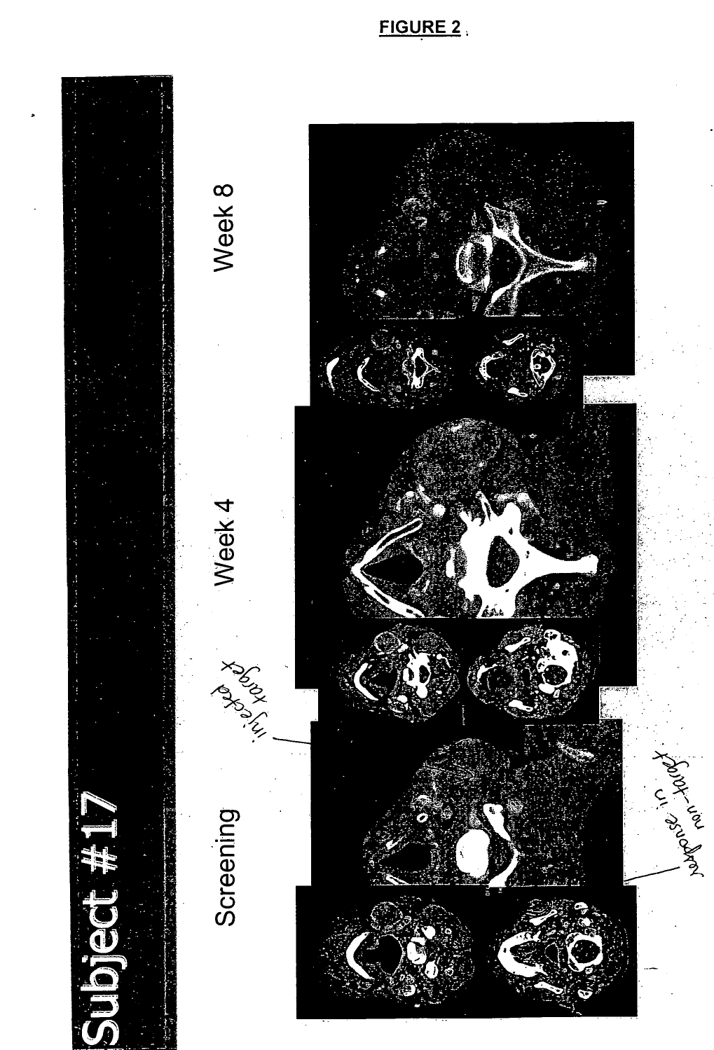

FIG. 3 shows a photograph (tumors not amenable to CT scan) of target lesion, nodal network lesions and distal nodes at screening, week 4 (end of 4 week dosing period) and week 8 (4 weeks after last dose) showing complete resolution target, nodal network an distal lesions.

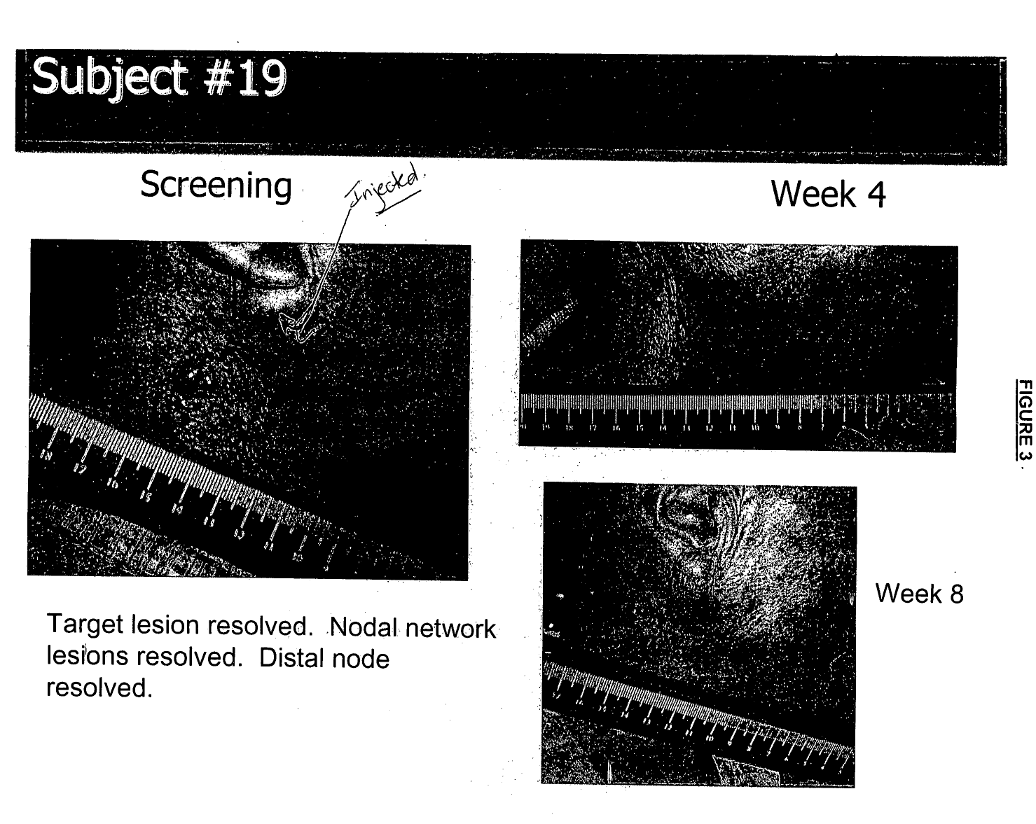

### DETAILED DESCRIPTION OF THE INVENTION

The present invention relates to an improved method to treat cancer. The inventors have injected an immunotoxin directly into the tumor of patients with squamous cell carcinoma of the head and neck. The results have shown treatment of both the primary as well as secondary tumors.

Accordingly, the present invention provides a method of improving the efficacy of a cancer therapeutic comprising administering the cancer therapeutic to a first cancer lesion wherein the first cancer lesion is connected through the lymphatic system to a second cancer lesion and wherein the cancer therapeutic can thereby travel to the second cancer lesion through the lymphatic system.

The “cancer therapeutic” can be any therapeutic agent that can be used to treat cancer. The cancer therapeutic is preferably an agent that recognizes a cell surface molecule that is largely specific for the tumor type to be treated and that is largely absent from normal cells, especially cells of the lymphatic vessels and nodes. In a preferred embodiment, the cancer therapeutic is an immunotoxin that comprises a) a ligand that can bind to the cancer cells linked to b) a toxin that is cytotoxic to the cancer cells. The ligand is preferably an antibody or antibody fragment that binds to a cancer-associated antigen. Antibody fragments that may be used in the immunotoxin include Fab, Fab′, F(ab′)₂, scFv and dsFv fragments from recombinant sources and/or produced in transgenic animals. The antibody or fragment may be from any species including mice, rats, rabbits, hamsters and humans. Chimeric antibody derivatives, i.e., antibody molecules that combine a non-human animal variable region and a human constant region are also contemplated within the scope of the invention. Chimeric antibody molecules can include, for example, humanized antibodies which comprise the antigen binding domain from an antibody of a mouse, rat, or other species, with human constant regions. Conventional methods may be used to make chimeric antibodies (see, for example, Morrison et al., Proc. Natl Acad. Sci. U.S.A. 81,6851 (1985); Takeda et al., Nature 314, 452 (1985); Cabilly et al., U.S. Pat. No. 4,816,567; Boss et al., U.S. Pat. No. 4,816,397; Tanaguchi et al., European Patent Publication EP171496; European Patent Publication 0173494, United Kingdom patent GB 2177096B). The preparation of humanized antibodies is described in EP-B 10 239400. Humanized antibodies can also be commercially produced (Scotgen Limited, 2 Holly Road, Twickenham, Middlesex, Great Britain). It is expected that chimeric antibodies would be less immunogenic in a human subject than the corresponding non-chimeric antibody. The humanized antibodies can be further stabilized for example as described in WO 00/61635 which is incorporated herein by reference.

In one embodiment, the cancer-associated antigen recognized by the immunotoxin is Ep-CAM. In a preferred embodiment, the immunotoxin comprises an antibody or antibody fragment that binds to Ep-CAM linked to Pseudomonas exotoxin A or a modified form thereof. In a specific embodiment, the immunotoxin is VB4-845 which is described in WO 00/61635 and WO 2004/096271 both of which are incorporated herein by reference.

It is preferable that the cancer therapeutic (such as an immunotoxin) not be toxic to non-cancer cells and not be “trapped” by the lymph node except if it binds to cancerous cells in the lymph node. Unbound immunotoxin or other treatment agent should remain available to travel along the lymphatic system and to bind to any rogue cancer cells. In this regard, it is preferable to use small ligands such as antibody fragments including single chain Fv (scFv), Fab, Fab′, F(ab′)₂ and dsFv regions in the immunotoxin.

The term “administering” the cancer therapeutic includes any and all modes of administration that can direct the cancer therapeutic to the first cancer lesion. This includes, but is not limited to, intra-tumoral, peri-tumoral or subcutaneous injection of the cancer therapeutic. “Administering” also includes providing the cancer therapeutic at or near the first cancer lesion in a slow release device or slow release formulation.

The phrase “improving the efficacy of a cancer therapeutic” as used herein means that the treatment of the cancer by the present invention results in an improved result as compared to administering a cancer therapeutic by another route such as systemic administration. An improvement in the efficacy of the treatment includes a greater inhibition of cancer cell replication, a greater inhibition of cancer spread (metastatic), a greater inhibition of tumor growth, a greater reduction of cancer cell number, a greater decrease in malignant grade of a cancer or an improvement in cancer related symptoms.

The cancer to be treated can be any type of cancer that can be accessed for intra-tumoral, peri-tumoral or subcutaneous injection and wherein the lesions are connected through the lymphatic system. This includes tumors of the head and neck, melanomas, breast tumors, stomach tumors and other tumors known to spread via the lymphatic system.

The “first cancer lesion” will be any cancer lesion that is connected to secondary lesions. The term “second cancer lesion” includes one or more secondary lesions that are connected to the first cancer lesion. The first cancer lesion should be selected based on it being ‘upstream’ of other cancerous lesions or potentially affected lymph nodes with regards to the lymphatic drainage. Typically, lymphatic drainage occurs towards the supraclavicular area, that is, lymphatic fluid drains more or less downwards for the head and neck area but fluid movement is upwards for areas below the shoulders. This general principle can be used to determine the lesion to be treated (i.e. which lesion is ‘upstream’) if lymphatic drainage mapping is not feasible. Although not wishing to be bound by any specific mechanism, it has been shown that tumors can direct the generation of new, intra-tumoral lymphatic vessels (lymphangiogenesis) and that these new vessels may be linked to the peri-tumoral vessels that then lead to other lesions (Oliver & Detmar, Genes and Development 16:773-783, 2002; Beasly et al., Cancer Res. 62: 1315-1320, 2002; Dadras et al., Amer. J. Pathol. 162: 1951-1960, 2003).

In one embodiment, the first cancer lesion can be selected by lymphatic drainage basin mapping to select a tumor that is linked to the largest number of secondary lesions. This is especially useful for cancer patients that present with multiple cancerous lesions as is seen, for example in cancers of the head and neck. Methods to map the lymphatic drainage basin are known to the art and include intra-tumoral, peri-tumoral or subcutaneous injection of radioactive tracer in the form of colloid, macromolecules or particles of a size that will favour their accumulation in the lymph nodes. Administration of blue dyes during surgery and once the area of interest has been exposed is also used, alone or in conjunction with lymphoscintigraphy to identify the vessels and associated nodes. (Takes, Oral Oncol. 40:656-657, 2004 {review of imaging techniques for nodal status determination in Head and Neck cancer}; Uren et al., J. Nucl. Med. 44: 570-582, 2003 {review of lymphoscintigraphy for melanomas}; Leidenius et al., Int. Surg. 87:160-163, 2002; Tanis et al., Annals Surg Oncol. 8:850-855, 2001 {lymphoscintigraphy for breast cancer}; Alex, Layngoscope 114:2-19, 2004 {radiolocalization and blue dye for various head and neck tumors}; Klutmann et al., J. Nucl. Med. 40:776-782; Ross et al., Brit. J. Radiol. 75:950-958, 2002 {double tracer method in SCCHN; Lymphsc. for SCCHN}. In addition to the commonly used lymphoscintigraphy and/or blue dye techniques, other lymphatic mapping methods have been described, including those using labelled antibodies (for eg U.S. Pat. Nos. 3,927,193; 4,460;561; 5,101,827), macromolecules (U.S. Pat. No. 5,582,172) or carbon black suspension (U.S. Pat. No. 6,815,170).

Accordingly, in one embodiment, the present invention provides a method of improving the efficacy of the cancer therapeutic comprising: a) conducting lymphatic drainage mapping on a patient; b) selecting a first cancer lesion that is linked through the lymphatic system to one or more second cancer lesions; and c) administering the cancer therapeutic to the first cancer lesion wherein the cancer therapeutic can travel to the second cancer lesions through the lymphatic system.

Lymphatic mapping can be done using an immunotoxin that comprises an agent (radio-opaque, radioactive etc) that allows for lymphatic mapping linked to a cancer-specific ligand. The ligand in the immunotoxin will guide the agent to the tumor site. The administration of such modified immunotoxin can occur prior to surgical treatment and the lymphatic map obtained can be used to guide either subsequent surgery or to modify the site of subsequent intratumoral injection. Lymphatic mapping can be omitted if the primary lesion can be determined based on clinical presentation and position relative to the expected lymphatic drainage pattern of the area. When the primary lesion is difficult to access, it would be possible to administer the agent in the tissue upstream in the lymphatic system such that the agent drains towards the lesion to be treated and secondary lesions.

In one embodiment, the methods of the present invention can be used prior to any other cancer treatment including radiation or therapy. In another embodiment, the methods of the present invention can be used after therapy such as surgery or radiation. Surprisingly, the inventors have shown that the approach is effective even when there has been significant damage to the tissues due to surgical dissection of cancerous tissue and associated healthy margins, radiation treatment and/or chemotherapy when it could expected that the lymphatic connection between individual tumors had been lost.

One of skill in the art will appreciate that the dose of the cancer therapeutic will depend on the type of cancer to be treated, the size of the tumor, the stage of the cancer, the frequency of administration as well as the age, weight, health of the patient. When using an immunotoxin as the cancer therapeutic the effective dose may preferably range from about 100 to 2000 μg per tumor, more preferably 300 to 1000 μg per tumor. The agent must be administered in excess of the binding capacity of the tumor in which it is injected. The excess can be achieved by means of a single injection and/or by repeated injections.

In one embodiment, the effective dose by direct administration of immunotoxin may range from about 10 to 3000, 20 to 900, 30 to 800, 40 to 700, 50 to 600, 60 to 500, 70 to 400, 80 to 300, 90 to 200, or 100 to 150 micrograms/tumor/day. In other embodiments, the dose may range from approximately 10 to 20, 21 to 40, 41 to 80, 81 to 100, 101 to 130, 131 to 150, 151 to 200, 201 to 280, 281 to 350, 351 to 500, 501 to 1000, 1001 to 2000, or 2001 to 3000 micrograms/tumor/day. In specific embodiments, the dose may be at least approximately 20, 40, 80, 130, 200, 280, 400, 500, 750, 1000, 2000, or 3000 micrograms/tumor/day.

In another embodiment, the effective dose of immunotoxin may range from about 100 to 5000, 200 to 4000, 300 to 3000, 400 to 2000, 500 to 1000, 600 to 900, or 700 to 1500 micrograms/tumor/month. In other embodiments, the dose may range from approximately 100 to 19,9, 200 to 399, 400 to 649, 650 to 999, 1000 to 1799, 1800 to 2499, 2500 to 3499, 3500 to 4999, 5000 to 7499, 7500 to 10000, or 10001 to 20000 micrograms/tumor/month. In specific embodiments, the dose may be at least approximately 100, 200, 400, 650, 1000, 1400, 2000, 2500, 3000, 3500, 4000, 4500, 5000, 7500, 10000, or 20000 micrograms/tumor/month.

In another embodiment, the effective dose of immunotoxin results in an intratumoral concentration of at least approximately 5, 10, 20, 30, 40, 50, 60, 75, 100, 125, 150, 100, 200, 300, 400, or 500 micrograms/cm³ of the immunotoxin. In other embodiments, the resulting intratumoral concentration of immunotoxin is approximately 5 to 500, 10 to 400, 15 to 300, 20 to 200, 25 to 100, 30 to 90, 35 to 80, 40 to 70, 45 to 60, or 50 to 55 micrograms/cm³. In other embodiments, the resulting intratumoral concentration of immunotoxin is approximately 10 to 15, 16 to 20, 21 to 25, 26 to 30, 31 to 35, 36 to 40, 41 to 45, 46 to 50, 51 to 55, 56 to 60, 61 to 65, 66 to 70, 71 to 75, 76 to 80, 81 to 85, 86 to 90, 91 to 95, 96 to 100, or 100 to 200 micrograms/cm³.

In another embodiment, the effective dose of immunotoxin results in a plasma concentration of less than approximately 0.1, 1, 2.5, 5, 7.5, 10, 15, 20, 30, 40, or 50 micrograms/liter. In other embodiments, the resulting circulating concentration of immunotoxin is approximately 0.1 to 50, 1 to 40, 2.5 to 30, 5 to 20, or 7.5 to 10 micrograms/liter. In other embodiments, the resulting circulating concentration of immunotoxin is approximately 0.1 to 1, 1.1 to 2.4, 2.5 to 5, 5.1 to 7.4, 7.5 to 10, 11 to 15, 16 to 20, 21 to 30, 31 to 40, or 41 to 50 micrograms/liter.

In a particular non-limiting embodiment, the effective dose of the immunotoxin is between about 100 and 3000 micrograms/tumor/month, for example approximately 100, 200, 300, 400, 750, or 1000 micrograms/tumor/month, wherein the patient is administered a single dose per day. The single dose is administered approximately every month for approximately 1, 2, 3, 4, 5, or 6 consecutive months. After this cycle, a subsequent cycle may begin approximately 1, 2, 4, 6, or 12 months later. The treatment regime may include 1, 2, 3, 4, 5, or 6 cycles, each cycle being spaced apart by approximately 1, 2, 4, 6, or 12 months.

In a particular non-limiting embodiment, the effective dose of the immunotoxin is between about 20 and 1240 micrograms/tumor/day, for example approximately 20, 40, 80, 130, 200, or 280 micrograms/tumor/day or approximately 100, 200, 330, 500, 700, 930, 1240 micrograms/tumor/day, wherein the patient is administered a single dose per day. The single dose is administered approximately every day (one or more days may optionally be skipped) for approximately 1, 2, 3, 4, 5, 6 or 7 consecutive days. After this cycle, a subsequent cycle may begin approximately 1, 2, 3, 4, 5, or 6 weeks later. The treatment regime may include 1, 2, 3, 4, 5, or 6 cycles, each cycle being spaced apart by approximately 1, 2, 3, 4, 5, or 6 weeks.

The injection volume preferably is at least an effective amount, which is appropriate to the type and/or location of the tumor. The maximum injection volume in a single dose may be between about 25% and 75% of tumor volume, for example approximately one-quarter, one-third, or three-quarters of the estimated target tumor volume. In a specific, non-limiting embodiment, the maximum injection volume in a single dose is approximately 30% of the tumor volume.

In another embodiment, the immunotoxin is administered intratumourally at a total dose per cycle equivalent to, or below the maximum tolerated dose established in a safety trial but the dosage is standardized in relation to the tumor volume. For example, subjects will receive between 1 microgram per cm³ and 500 microgram per cm³ tumor or a dose sufficient to reach about between 14 picomole and 7 nanomole per cm³ tumor tissue. The dose will be administered in a volume not exceeding about 20-50% of the tumor volume. The immunotoxin will be diluted in a suitable salt solution. For example, for a tumor of estimated volume of 3 cm³, a target dose of 14 picomoles (1 microgram per cm³), and a maximum injection relative volume of about ⅓ of the tumor, 3 microgram of immunotoxin will be diluted into about 1 ml of diluent.

In another particular embodiment, the effective dose of the immunotoxin is between about 20 and 300 micrograms/tumor/day, for example approximately 20, 40, 80, 130, 200, or 280 micrograms/tumor/day, wherein the patient is administered a single dose per day. The maximum injection volume in a single dose may be between about 25% and 75% of tumor volume, for example approximately one-quarter, one-third, or three-quarters of the estimated target tumor volume. The single dose is administered every other day for approximately 5, 7, 9, 11, 13, 15, 17, 19, 21, 23, 25, 27, 29, or 31 consecutive days. After this cycle, a subsequent cycle may begin approximately 1, 2, 3, 4, 5, 6, 7, 8, 9, 10, 11, or 12 weeks later. The treatment regime may include 1, 2, 3, 4, 5, or 6 cycles, each cycle being spaced apart by approximately 1, 2, 3, 4, 5, 6, 7, 8, 9, 10, 11, or 12 weeks.

In one specific non-limiting embodiment, VB4-845 is administered at a dose of approximately 280 micrograms/tumor/day, wherein the patient is administered a single dose per day. The maximum injection volume in a single dose is approximately one-third of the estimated target tumor volume. The single dose is administered every day for approximately five consecutive days. After this cycle, a subsequent cycle may begin approximately one month later, preferably one month from the first day of the first cycle. The treatment regime may include three cycles, each cycle being spaced apart by approximately one treatment-free week.

In another specific non-limiting embodiment, VB4-845 is administered at a dose of approximately 280 micrograms/tumor/day, wherein the patient is administered a single dose per day. The maximum injection volume in a single dose is approximately one-third of the estimated target tumor volume. The single dose is administered every other day for approximately one week. After this cycle, a subsequent cycle may begin approximately one week later. The treatment regime may include three cycles, each cycle being spaced apart by approximately one week.

In yet another specific embodiment, VB4-845 is administered at a dose of approximately 280 micrograms/tumor/day, wherein the patient is administered a single dose per day. The maximum injection volume in a single dose is approximately one-third of the estimated target tumor volume. The single dose is administered every other day for approximately three weeks. After this cycle, a subsequent cycle may begin approximately one week later. The treatment regime may include three cycles, each cycle being spaced apart by approximately one week. Further information on doses can be found in WO 2004/096271 which is incorporated herein by reference.

The present invention also includes kits for carrying out the methods of the inventions. In one embodiment, the kit comprises a cancer therapeutic (such as an immunotoxin) together with instructions for administering to the first cancer lesion. In another embodiment, the kit comprises a cancer therapeutic (such as an immunotoxin) together with instructions for identifying the first cancer lesion. In such an embodiment, the instructions can include instructions for lymphatic mapping as described above.

In a preferred embodiment, the kits of the invention comprise an immunotoxin as the cancer therapeutic. Preferably, the immunotoxin comprises an antibody or antibody fragment that binds to Ep-CAM linked to Pseudomonas exotoxin-A or a modified form thereof. More preferably, the immunotoxin is VB4-845 as described herein.

The following non-limiting example is illustrative of the present invention:

### EXAMPLE 1

In a dose escalating, phase I clinical trial in recurrent and refractory squamous cell carcinoma of the head and neck (SCCHN), accessible tumors were injected with an immunotoxin comprising an antigen-binding domain specific for Ep-CAM, in a single chain format (scFv) to which a modified form of Pseudomonas exotoxin A has been genetically attached. This immunotoxin known as VB4-845 has been described in part (scFv) or fully in WO 00/61635 and WO 2004/096271, respectively. Ep-CAM positivity of the tumor was evaluated by fine needle biopsy. Subjects had previously received various treatments, including one or more of surgery, radiation and chemotherapy. VB4-845 was administered intratumorally, once per week for 4 consecutive weeks. Subjects in each dosages groups received either 100, 200, 330, 500, 700 or 930 microgram per dose (injected into one and always the same lesion). A follow-up visit occurred 4 weeks after the last dosing. The dosage was not adjusted based on the size of the tumor and, thus, concentration of VB4-845 (mg/cm³ tumor) varied not only across patients within a dose group but, also over time if the tumor either regressed or progressed. Tumor response was evaluated based on the size of the tumor as determined at physical examination and by CT scan.

Despite the lack of optimization of the phase I trial design with respect to the evaluation of efficacy, many subjects responded to the treatment. Of the 20 patients entered and treated in the trial, 2 were Ep-CAM negative and did not show a response to treatment. Of the 18 Ep-CAM positive patients, 10 ‘response’ to ‘complete’ response of the treated tumor were observed. Of those at least 3 also showed clear response of secondary tumors. Subject information and results are shown in Tables 1 and 2, respectively. Table 2 also identifies the Figure number where the response is documented either by CT scan or by photographs.

While the present invention has been described with reference to what are presently considered to be the preferred examples, it is to be understood that the invention is not limited to the disclosed examples. To the contrary, the invention is intended to cover various modifications and equivalent arrangements included within the spirit and scope of the appended claims.

All publications, patents and patent applications are herein incorporated by reference in their entirety to the same extent as if each individual publication, patent or patent application was specifically and individually indicated to be incorporated by reference in its entirety.

TABLE 1
Patient Information

| Pa- | | | Lesion | Dose | |
|---|---|---|---|---|---|
| tient | | Lesion | Volume | mg/cm³ | |
| No | EpCAM | Location | cm³ | (mg) | Prior Treatments |
| # 7 | 3+ | Left | 2.70 | 0.122 | S: Hemiglossectomy, |
| | (75%) | Cervical | | (.330) | Pharyngolaryngectomy, |
| | | LN | | | Tracheotomy |
| | | | | | Rad: Yes |
| | | | | | Chemo: Cisplatin + |
| | | | | | 5-FU; Methotrexate |
| # 17 | 3+ | Left | 4.00 | 0.233 | S: No |
| | (15%) | Neck LN | | (.930) | Rad: Yes |
| | | | | | Chemo: Carbo/Taxol; |
| | | | | | Cisplatin |
| # 19 | 2+ | Right | 0.55 | 1.691 | S: Laryngectomy |
| | (30%) | Subman- | | (.930) | Rad: Yes |
| | | dibular | | | Chemo: No |

NOTES:
Lesion Volume: at screening
Dose in mg/cm³: based on lesion volume at screening
Dose in mg: total amount of immunotoxin drug injected
EpCAM Staining intensity: 0 = Trace/Faint; 1+ = Weak; 2+ = Moderate; 3+ = Strong
(%); indicates percentage of cells in fine needle aspirate that stained positive - may not be representative of whole tumor.
ABBREVIATIONS:
S = Surgery;
Rad = Radiation;
Chemo = Chemotherapy;
LN = Lymph Node

TABLE 2
Response to Intratumoral Immunotoxin

| | | | Dose | |
|---|---|---|---|---|
| Patient | | Figure | mg/cm³ | |
| No. | EpCAM | No. | (mg) | Response |
| # 7 | 3+ | 1a, 1b | 0.122 | TL: Completely Regressed |
| | (75%) | | (.330) | N-TL: Large deep mass |
| | | | | decreased in size (partial |
| | | | | response) |
| # 17 | 3+ | 2 | 0.233 | TL: Stable |
| | (15%) | | (.930) | N-TL: Complete regression |
| | | | | of secondary lymph node; partial |
| | | | | response other lymph nodes |
| # 19 | 2+ | 3 | 1.691 | TL: Resolved |
| | (30%) | | (.930) | N-TL: Nodal lesions resolved; |
| | | | | distal nodes resolved |

NOTES:
Dose in mg/cm³: based on lesion volume at screening
Dose in mg: total amount of immunotoxin drug injected
EpCAM Staining intensity: 0 = Trace/Faint; 1+ = Weak; 2+ = Moderate; 3+ = Strong
(%); indicates percentage of cells in fine needle aspirate that stained positive - may not be representative of whole tumor.
ABBREVIATIONS:
TL = Target lesion;
N-TL = Non-target lesion

1. A method of improving the efficacy of a cancer therapeutic comprising administering the cancer therapeutic to a first cancer lesion wherein the first cancer lesion is connected through the lymphatic system to a second cancer lesion and wherein the cancer therapeutic can thereby travel to the second cancer lesion through the lymphatic system.

2. A method of improving the efficacy of the cancer therapeutic according to claim 1 comprising:
a) conducting lymphatic drainage mapping on a patient;
b) selecting a first cancer lesion that is linked through the lymphatic system to one or more second cancer lesions; and
c) administering the cancer therapeutic to the first cancer lesion wherein the cancer therapeutic can travel to the second cancer lesions through the lymphatic system.

3. A method according to claim 1 wherein the cancer therapeutic is an immunotoxin.

4. A method according to claim 2 wherein the cancer therapeutic is an immunotoxin.

5. A method according to claim 3 wherein the immunotoxin comprises an antibody or antibody fragment that binds to Ep-CAM linked to a toxin.

6. A method according to claim 2 wherein the immunotoxin comprises an antibody or antibody fragment that binds to Ep-CAM linked to a toxin.

7. A method according to claim 5 wherein the toxin is derived from Pseudomonas exotoxin.

8. A method according to claim 6 wherein the toxin is derived from Pseudomonas exotoxin.

9. A method according to claim 5 wherein the antibody fragment is a single chain Fv (scFv).

10. A method according to claim 1 wherein the cancer therapeutic is administered intra-tumorally.

11. A method according to claim 2 wherein the cancer therapeutic is administered intra-tumorally.

12. A method according to claim 5 wherein the cancer therapeutic is administered intra-tumorally.

13. A method according to claim 1 wherein the first cancer lesion is a cancer of the head and neck.

14. A method according to claim 2 wherein the first cancer lesion is a cancer of the head and neck.

15. A method according to claim 5 wherein the first cancer lesion is a cancer of the head and neck.

16. A method according to claim 15 wherein the immunotoxin is administered in a dose from about 100 to 2000 μg per tumor.

17. A kit for conducting the method of claim 1 comprising the cancer therapeutic together with instructions for administering to the first cancer lesion.

18. A kit for conducting the method of. claim 2 comprising the cancer therapeutic together with instructions for identifying the first cancer lesion.

19. A kit according to claim 18 wherein the instructions comprise instructions for lymphatic drainage basin mapping.
